(19) United States
(12) Patent Application Publication
(10) Pub. No.: US 2021/0343028 A1
(43) Pub. Date: Nov. 4, 2021

(54) ANALYSIS DEVICE, ANALYSIS METHOD, NON-TRANSIENT COMPUTER-READABLE RECORDING MEDIUM STORED WITH PROGRAM, AND CALIBRATION METHOD

(71) Applicant: Honda Motor Co., Ltd., Tokyo (JP)

(72) Inventors: Yasushi IKEUCHI, Saitama (JP); Haruo AOKI, Saitama (JP); Jun ASHIHARA, Saitama (JP); Masayo ARAI, Saitama (JP); Takeshi OSATO, Saitama (JP); Kenichi TOYA, Saitama (JP); Yousuke NAGATA, Saitama (JP); Taizo YOSHIKAWA, Saitama (JP)

(73) Assignee: Honda Motor Co., Ltd., Tokyo (JP)

(21) Appl. No.: 17/242,330

(22) Filed: Apr. 28, 2021

(30) Foreign Application Priority Data
Apr. 30, 2020 (JP) 2020-080278

(51) Int. Cl. G06T 7/246; G06K 9/00; G06T 7/80; G06K 9/62; G01P 15/18; G01P 3/00; G01P 15/08

(52) U.S. Cl. CPC G06T 7/251; G06K 9/00342; G06T 7/80; G06K 9/6288; G06T 2207/30196; G01P 3/00; G01P 15/08; G06T 2207/30208; G01P 15/18

(57) ABSTRACT

Provided are an analysis device, an analysis method, a program, and a calibration method. The analysis device includes: an obtaining part obtaining an image captured by an image capturing part that captures an image of one or more first markers provided on an estimation target; and a calibration part calibrating a conversion rule from a sensor coordinate system to a segment coordinate system based on the image. A posture of the first marker relative to at least one inertial measurement sensor does not change, and the posture with respect to the image capturing part is recognizable by analyzing the captured image. The calibration part derives the posture of the first marker with respect to the image capturing part, derives a conversion matrix from the sensor coordinate system to a camera coordinate system based on the derived posture, and calibrates the conversion rule by using the derived conversion matrix.

### CROSS-REFERENCE TO RELATED APPLICATION

This application claims the priority benefit of Japan application serial no. 2020-080278, filed on Apr. 30, 2020. The entirety of the above-mentioned patent application is hereby incorporated by reference herein and made a part of this specification.

### TECHNICAL FIELD

The disclosure relates to an analysis device, an analysis method, a non-transient computer-readable recording medium stored with a program, and a calibration method.

### DESCRIPTION OF RELATED ART

Conventionally, a technique (motion capture) for estimating the body posture and its change (motion) by attaching to the body multiple inertial measurement unit (IMU) sensors capable of measuring the angular velocity and the acceleration has been disclosed (see, for example, Patent Document 1).
[Patent Document 1] Japanese Laid-open No. 2020-42476

In the estimation technique by using the IMU sensor, calibration may be performed for the rule of converting the output of the IMU sensor into a certain coordinate system in the initial posture when the IMU sensor is attached to the body of the subject. However, depending on the subsequent movement of the subject, after the IMU sensor is calibrated, the attachment position and posture of the IMU sensor may change from the time of calibration, and the conversion rule may not be appropriate.

### SUMMARY

The analysis device, the analysis method, the non-transient computer-readable recording medium stored with the program, and the calibration method according to the disclosure adopt the following configurations.

(1) An analysis device according to an aspect of the disclosure includes: a posture estimation part which estimates a posture of an estimation target including a process of converting an output of multiple inertial measurement sensors expressed in a sensor coordinate system based on respective positions of the inertial measurement sensors that are attached to multiple sites of the estimation target and detect angular velocity and acceleration into a segment coordinate system expressing postures of respective segments corresponding to the positions where the inertial measurement sensors are attached in the estimation target; an obtaining part which obtains an image captured by an image capturing part that captures an image of one or more first markers provided on the estimation target; and a calibration part which calibrates a conversion rule from the sensor coordinate system to the segment coordinate system based on the image. The first marker has a form in which a posture relative to at least one of the inertial measurement sensors does not change, and the posture with respect to the image capturing part is recognizable by analyzing the captured image. The calibration part derives the posture of the first marker with respect to the image capturing part, derives a conversion matrix from the sensor coordinate system to a camera coordinate system based on the derived posture, and calibrates the conversion rule from the sensor coordinate system to the segment coordinate system by using the derived conversion matrix from the sensor coordinate system to the camera coordinate system.

### BRIEF DESCRIPTION OF THE DRAWINGS

FIG. 1 is a diagram showing an example of a usage environment of an analysis device 100.

FIG. 2 is a diagram showing an example of disposition of the IMU sensors 40.

FIG. 3 is a diagram showing an example of a more detailed configuration and function of the posture estimation part 120.

FIG. 4 is a diagram for illustrating a plane assumption process by the correction part 160.

FIG. 5 is a diagram for illustrating a definition process of a direction vector vi by the correction part 160.

FIG. 6 is a diagram showing a state in which the direction vector vi is swiveled due to a change in the posture of the estimation target TGT.

FIG. 7 is a diagram for schematically illustrating the correction process by the analysis device 100.

FIG. 8 is a diagram showing an example of the configuration of the whole body correction amount calculation part 164.

FIG. 9 is a diagram showing another example of the configuration of the whole body correction amount calculation part 164.

FIG. 10 is a diagram schematically showing the overall process of the whole body correction amount calculation part 164.

FIG. 11 is a diagram for stepwise illustrating the flow of the process of the whole body correction amount calculation part 164.

FIG. 12 is a diagram for stepwise illustrating the flow of the process of the whole body correction amount calculation part 164.

FIG. 13 is a diagram for stepwise illustrating the flow of the process of the whole body correction amount calculation part 164.

FIG. 14 is a diagram showing an example of the appearance of the first marker Mk1.

FIG. 15 is a diagram showing an example of a captured image IM1.

FIG. 16 is a diagram for illustrating the content of the process by the calibration part 180.

FIG. 17 is a diagram showing an example of a captured image IM2.

FIG. 18 is a diagram for illustrating a (first) modified example of the method of obtaining the captured image.

FIG. 19 is a diagram for illustrating a (second) modified example of the method of obtaining the captured image.

### DESCRIPTION OF THE EMBODIMENTS

The disclosure has been made in consideration of such circumstances, and the disclosure provides an analysis device, an analysis method, a program, and a calibration method capable of appropriately performing calibration related to posture estimation by using an IMU sensor.

The analysis device, the analysis method, the non-transient computer-readable recording medium stored with the program, and the calibration method according to the disclosure adopt the following configurations.

(1) An analysis device according to an aspect of the disclosure includes: a posture estimation part which estimates a posture of an estimation target including a process of converting an output of multiple inertial measurement sensors expressed in a sensor coordinate system based on respective positions of the inertial measurement sensors that are attached to multiple sites of the estimation target and detect angular velocity and acceleration into a segment coordinate system expressing postures of respective segments corresponding to the positions where the inertial measurement sensors are attached in the estimation target; an obtaining part which obtains an image captured by an image capturing part that captures an image of one or more first markers provided on the estimation target; and a calibration part which calibrates a conversion rule from the sensor coordinate system to the segment coordinate system based on the image. The first marker has a form in which a posture relative to at least one of the inertial measurement sensors does not change, and the posture with respect to the image capturing part is recognizable by analyzing the captured image. The calibration part derives the posture of the first marker with respect to the image capturing part, derives a conversion matrix from the sensor coordinate system to a camera coordinate system based on the derived posture, and calibrates the conversion rule from the sensor coordinate system to the segment coordinate system by using the derived conversion matrix from the sensor coordinate system to the camera coordinate system.

(2) In the above aspect (1), the image capturing part further captures an image of a second marker which is stationary in a space where the estimation target is present; the second marker has a form in which a posture with respect to the image capturing part is recognizable by analyzing the captured image; and the calibration part derives the posture of the second marker with respect to the image capturing part, derives a conversion matrix from a global coordinate system expressing the space to the camera coordinate system based on the derived posture, and equates the segment coordinate system with the global coordinate system, whereby the calibration part derives a conversion matrix from the sensor coordinate system to the segment coordinate system based on the conversion matrix from the sensor coordinate system to the camera coordinate system and the conversion matrix from the global coordinate system to the camera coordinate system and calibrates the conversion rule from the sensor coordinate system to the segment coordinate system based on the derived conversion matrix from the sensor coordinate system to the segment coordinate system.

(3) In the above aspect (1) or aspect (2), the image capturing part further captures an image of a third marker which is provided on the estimation target; the third marker has a form in which a posture relative to at least one of the segments does not change, and the posture with respect to the image capturing part is recognizable by analyzing the captured image; and the calibration part derives the posture of the third marker with respect to the image capturing part, derives a conversion matrix from the segment coordinate system to the camera coordinate system based on the derived posture, derives a conversion matrix from the sensor coordinate system to the segment coordinate system based on the conversion matrix from the sensor coordinate system to the camera coordinate system and the conversion matrix from the segment coordinate system to the camera coordinate system and calibrates the conversion rule from the sensor coordinate system to the segment coordinate system based on the derived conversion matrix from the sensor coordinate system to the segment coordinate system.

(4) In an analysis method according to another aspect of the disclosure, a computer performs: estimating a posture of an estimation target including a process of converting an output of a plurality of inertial measurement sensors expressed in a sensor coordinate system based on respective positions of the inertial measurement sensors that are attached to a plurality of sites of the estimation target and detect angular velocity and acceleration into a segment coordinate system expressing postures of respective segments corresponding to the positions where the inertial measurement sensors are attached in the estimation target; obtaining an image captured by an image capturing part that captures an image of one or more first markers provided on the estimation target; and calibrating a conversion rule from the sensor coordinate system to the segment coordinate system based on the image. The first marker has a form in which a posture relative to at least one of the inertial measurement sensors does not change, and the posture with respect to the image capturing part is recognizable by analyzing the captured image. In the process of calibrating, the computer derives the posture of the first marker with respect to the image capturing part, derives a conversion matrix from the sensor coordinate system to a camera coordinate system based on the derived posture, and calibrates the conversion rule from the sensor coordinate system to the segment coordinate system by using the derived conversion matrix from the sensor coordinate system to the camera coordinate system.

(5) The non-transient computer-readable recording medium stored with the program according to another aspect of the disclosure makes a computer perform: estimating a posture of an estimation target including a process of converting an output of a plurality of inertial measurement sensors expressed in a sensor coordinate system based on respective positions of the inertial measurement sensors that are attached to a plurality of sites of the estimation target and detect angular velocity and acceleration into a segment coordinate system expressing postures of respective segments corresponding to the positions where the inertial measurement sensors are attached in the estimation target; obtaining an image captured by an image capturing part that captures an image of one or more first markers provided on the estimation target; and calibrating a conversion rule from the sensor coordinate system to the segment coordinate system based on the image. The first marker has a form in which a posture relative to at least one of the inertial measurement sensors does not change, and the posture with respect to the image capturing part is recognizable by analyzing the captured image. In the process of calibrating, the computer derives the posture of the first marker with respect to the image capturing part, derives a conversion matrix from the sensor coordinate system to a camera coordinate system based on the derived posture, and calibrates the conversion rule from the sensor coordinate system to the segment coordinate system by using the derived conversion matrix from the sensor coordinate system to the camera coordinate system.

(6) A calibration method according to another aspect of the disclosure includes: capturing an image of the one or more first markers provided on the estimation target by the image capturing part equipped on an unmanned aerial vehicle; and obtaining the image captured by the image capturing part and calibrating the conversion rule from the sensor coordinate system to the segment coordinate system by the analysis device according to any one of aspects (1) to (3).

(7) A calibration method according to another aspect of the disclosure includes: capturing an image of the one or more first markers provided on the estimation target by the image capturing part attached to a stationary object; and obtaining the image captured by the image capturing part and calibrating the conversion rule from the sensor coordinate system to the segment coordinate system by the analysis device according to any one of aspects (1) to (3).

(8) A calibration method according to another aspect of the disclosure includes: capturing an image of the one or more first markers provided on the estimation target by the image capturing part attached to the estimation target; and obtaining the image captured by the image capturing part and calibrating the conversion rule from the sensor coordinate system to the segment coordinate system by the analysis device according to any one of aspects (1) to (3).

According to the above aspects (1) to (8), the IMU sensors can be appropriately calibrated.

Hereinafter, embodiments of the analysis device, analysis method, program, and calibration method of the disclosure will be described with reference to the drawings.

The analysis device is realized by at least one processor. The analysis device is, for example, a service server which communicates with a user's terminal device via a network. Alternatively, the analysis device may be a terminal device in which an application program is installed. In the following description, it is assumed that the analysis device is a service server.

The analysis device is a device which obtains detection results from multiple inertial sensors (IMU sensors) attached to an estimation target such as a human body, and estimates a posture of the estimation target and the like based on the detection results. The estimation target is not limited to the human body as long as it includes segments (which may be regarded as rigid bodies in analytical mechanics, such as arms, hands, legs, and feet, in other words, links) and joints which connect two or more segments. That is, the estimation target is a human being, an animal, or a robot having a limited motion range of joints.

### First Embodiment

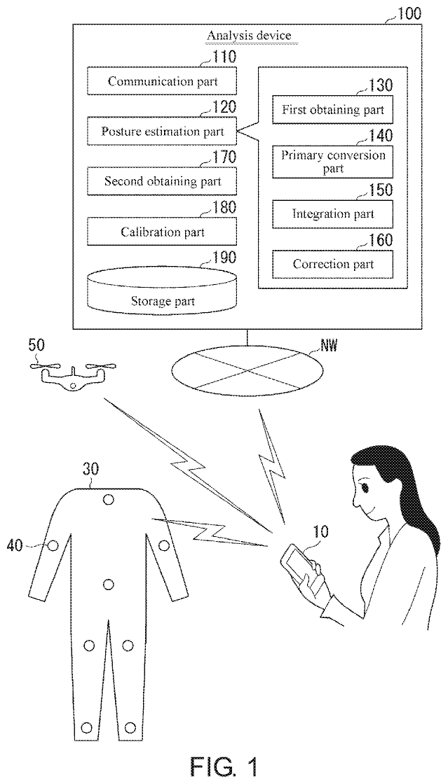

FIG. 1 is a diagram showing an example of a usage environment of an analysis device 100. A terminal device 10 is a smartphone, a tablet terminal, a personal computer, or the like. The terminal device 10 communicates with the analysis device 100 via a network NW. The network NW includes a wide area network (WAN), a local area network (LAN), the Internet, a cellular network, and the like. An image capturing device 50 is, for example, an unmanned aerial vehicle (drone) equipped with an image capturing part (camera). The image capturing device 50 is operated by, for example, the terminal device 10, and transmits a captured image to the analysis device 100 via the terminal device 10. The image captured by the image capturing device 50 is used by a calibration part 180. This will be described later.

IMU sensors 40 are attached to, for example, a measurement wear 30 worn by a user who is the estimation target. The measurement wear 30 is, for example, a wear in which multiple IMU sensors 40 are attached to easy-to-move clothes for sports. Further, the measurement wear 30 may be a wear in which multiple IMU sensors 40 are attached to a simple wearing piece such as a rubber band, a swimsuit, or a supporter.

The IMU sensor 40 is, for example, a sensor which detects acceleration and angular velocity for each of the three axes. The IMU sensor 40 includes a communication device, and transmits the acceleration and the angular velocity detected in cooperation with an application to the terminal device 10 by wireless communication. When the measurement wear 30 is worn by the user, which part of the user's body each of the IMU sensors 40 corresponds to (hereinafter referred to as disposition information) is naturally determined.

[Regarding Analysis Device 100]

The analysis device 100 includes, for example, a communication part 110, a posture estimation part 120, a second obtaining part 170, and a calibration part 180. The posture estimation part 120 includes, for example, a first obtaining part 130, a primary conversion part 140, an integration part 150, and a correction part 160. These components are realized by, for example, a hardware processor such as a central processing unit (CPU) executing a program (software). Some or all of these components may be realized by hardware (including a circuit part or a circuitry), such as a large scale integration (LSI), an application specific integrated circuit (ASIC), a field-programmable gate array (FPGA), a graphics processing unit (GPU), or may be realized by the cooperation of software and hardware. A program may be stored in advance in a storage device (a storage device including a non-transient storage medium) such as a hard disk drive (HDD) or a flash memory, or may be stored in a removable storage medium (non-transient storage medium) such as a DVD or a CD-ROM and installed by mounting the storage medium in a drive device. Further, the analysis device 100 includes a storage part 190. The storage part 190 is realized by an HDD, a flash memory, a random access memory (RAM), or the like.

The communication part 110 is a communication interface such as a network card for accessing the network NW.

[Posture Estimation Process]

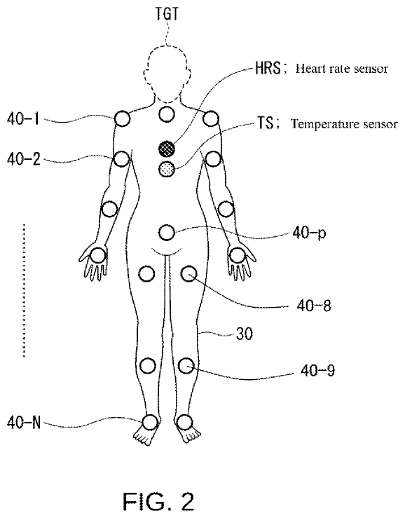

Hereinafter, an example of the posture estimation process by the posture estimation part 120 will be described. FIG. 2 is a diagram showing an example of disposition of the IMU sensors 40. For example, the IMU sensors 40-1 to 40-N (N is the total number of the IMU sensors) are attached to multiple sites such as the user's head, chest, pelvis area, and left and right limbs. In the following description, the user who wears the measurement wear 30 may be referred to as the estimation target TGT. Further, an argument i is used to mean any of 1 to N, and is referred to as the IMU sensor 40-i or the like. In the example of FIG. 2, a heart rate sensor and a temperature sensor are also attached to the measurement wear 30.

For example, the IMU sensor 40-1 is disposed on the right shoulder; the IMU sensor 40-2 is disposed on the upper right arm; the IMU sensor 40-8 is disposed on the left thigh; the IMU sensor 40-9 is disposed on the lower left knee, and so on; the IMU sensors 40 are disposed in this way. Further, the IMU sensor 40-p is attached near a site serving as a basis site. The basis site corresponds to, for example, a part of the trunk such as the user's pelvis. In the following description, a target site to which one or more IMU sensors 40 are attached and whose movement is measured is referred to as a “segment.” The segments include a basis site and a sensor attachment site (hereinafter referred to as a reference site) other than the basis site.

In the following description, the components corresponding to each of the IMU sensors 40-1 to 40-N will be described with the reference numeral followed by a hyphen and a reference numeral.

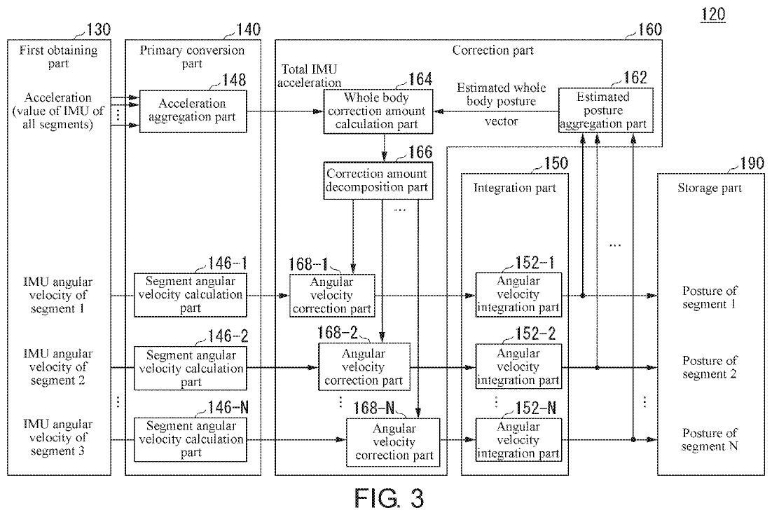

FIG. 3 is a diagram showing an example of a more detailed configuration and function of the posture estimation part 120. The first obtaining part 130 obtains information of the angular velocity and the acceleration from the multiple IMU sensors 40. The primary conversion part 140 converts the information obtained by the first obtaining part 130 from a three-axial coordinate system in each of the IMU sensors 40 (hereinafter referred to as the sensor coordinate system) into information of the segment coordinate system, and outputs the conversion results to the correction part 160.

The primary conversion part 140 includes, for example, a segment angular velocity calculation part 146-i corresponding to each segment and an acceleration aggregation part 148. The segment angular velocity calculation part 146-i converts the angular velocity of the IMU sensor 40-i output by the first obtaining part 130 into information of the segment coordinate system. The segment coordinate system is a coordinate system that expresses the posture of each segment. The process result (based on the detection results of the IMU sensors 40 and expressing the posture of the estimation target TGT) by the segment angular velocity calculation part 146-i is stored in the form of a quaternion, for example. Further, the expression of the measurement result of the IMU sensor 40-i in the form of a quaternion only serves as an example, and other expression methods such as a rotation matrix of a three-dimensional rotation group SO3 may be used.

The acceleration aggregation part 148 aggregates each acceleration detected by the IMU sensor 40-i corresponding to the segment. The acceleration aggregation part 148 converts the aggregation result into the acceleration of the whole body of the estimation target TGT (hereinafter, this may be referred to as the total IMU acceleration).

The integration part 150 integrates the angular velocity corresponding to the segment converted into the information of the basis coordinate system by the segment angular velocity calculation part 146-i to calculate the orientation of the segment to which the IMU sensor 40-i is attached in the estimation target TGT as a part of the posture of the estimation target. The integration part 150 outputs the integration results to the correction part 160 and the storage part 190.

Further, when the process cycle is the first time, the angular velocity output by the primary conversion part 140 (the angular velocity not corrected by the correction part 160) is input to the integration part 150, and subsequently, the angular velocity reflecting the correction derived based on the process result in the previous process cycle is input by the correction part 160, which will be described later.

The integration part 150 includes, for example, an angular velocity integration part 152-i corresponding to each segment. The angular velocity integration part 152-i integrates the angular velocity of the segment output by the segment angular velocity calculation part 146-i to calculate the orientation of the reference site to which the IMU sensor 40-i is attached in the estimation target as a part of the posture of the estimation target.

The correction part 160 assumes a representative plane passing through the basis site included in the estimation target, and corrects the converted angular velocity of the reference site so that the normal line of the representative plane and the orientation of the reference site calculated by the integration part 150 approach the directions orthogonal to each other. The representative plane will be described later.

The correction part 160 includes, for example, an estimated posture aggregation part 162, a whole body correction amount calculation part 164, a correction amount decomposition part 166, and an angular velocity correction part 168-i corresponding to each segment.

The estimated posture aggregation part 162 aggregates the quaternions expressing the posture of each segment, which are the calculation results by the angular velocity integration parts 152-i, into one vector. Hereinafter, the aggregated vector is referred to as the estimated whole body posture vector.

The whole body correction amount calculation part 164 calculates the correction amount of the angular velocity of all segments based on the total IMU acceleration output by the acceleration aggregation part 148 and the estimated whole body posture vector output by the estimated posture aggregation part 162. Further, the correction amount calculated by the whole body correction amount calculation part 164 is adjusted in consideration of the relationship between the segments so as not to be unnatural for the whole body posture of the estimation target. The whole body correction amount calculation part 164 outputs the calculation result to the correction amount decomposition part 166.

The correction amount decomposition part 166 decomposes the correction amount calculated by the whole body correction amount calculation part 164 into the correction amount of the angular velocity for each segment so that it may be reflected in the angular velocity of each segment. The correction amount decomposition part 166 outputs the decomposed correction amount of the angular velocity for each segment to the angular velocity correction part 168-i of the corresponding segment.

The angular velocity correction part 168-i reflects the decomposition result of the correction amount of the angular velocity of the corresponding segment output by the correction amount decomposition part 166 in the calculation result of the angular velocity for each segment output by the segment angular velocity calculation part 146-i. In this way, in the process of the next cycle, the target to be integrated by the integration part 150 becomes the angular velocity in the state in which the correction by the correction part 160 is reflected. The angular velocity correction part 168-i outputs the correction result to the angular velocity integration part 152-i.

The estimation result of the posture for each segment, which is the integration result by the integration part 150, is transmitted to the terminal device 10.

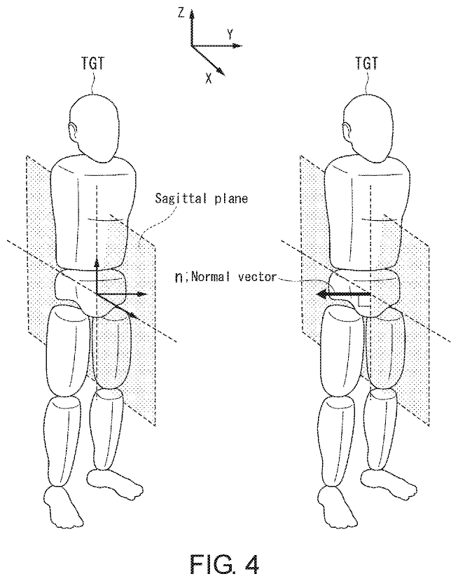

FIG. 4 is a diagram for illustrating a plane assumption process by the correction part 160. As shown in the left figure of FIG. 4, the correction part 160 assumes that a sagittal plane (“Sagittal plane” in the figure) passing through the center of the pelvis is the representative plane in the case where the basis site is the pelvis of the estimation target TGT. The sagittal plane is a plane which divides the body into left and right parts parallel to the midline of the body of the estimation target TGT that is bilaterally symmetrical. Further, the correction part 160 sets a normal line n of the assumed sagittal plane (arrow “Normal vector” in the figure) as shown in the right figure of FIG. 4.

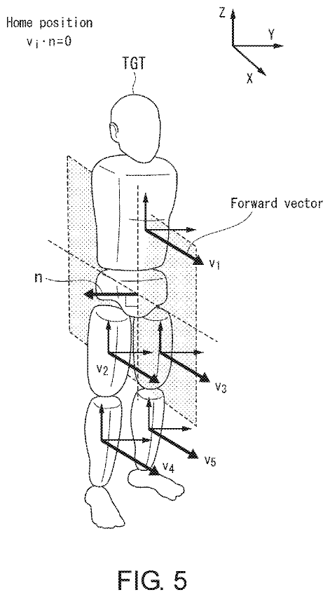

FIG. 5 is a diagram for illustrating a definition process of a direction vector vi by the correction part 160. The correction part 160 defines the output of a certain IMU sensor 40-i as an initial state, and defines the orientation as horizontal and parallel to the representative plane (first calibration process). After that, the direction vector is swiveled in three directions along the rotation in the three directions obtained by integrating the output of the IMU sensor 40-i.

As shown in FIG. 5, in the case where the reference site of the estimation target TGT includes the chest, the left and right thighs, and the left and right lower knees, the correction part 160 estimates the attachment postures of the IMU sensors 40 based on the result of the first calibration process, and corrects each of the converted angular velocities of the reference sites so that the normal line n and the orientations of the reference sites calculated by the integration part 150 approach the directions orthogonal to each other, and derives direction vectors v1 to v5 (“Forward vector” in the figure) facing the reference sites as shown in the figure. As shown in the figure, the direction vector v1 shows the direction vector of the chest; the direction vectors v2 and v3 show the direction vectors of the thighs; and the direction vectors v4 and v5 show the direction vectors of the lower knees. Further, the x axis, y axis, and z axis in the figure show an example of the directions of the basis coordinate system.

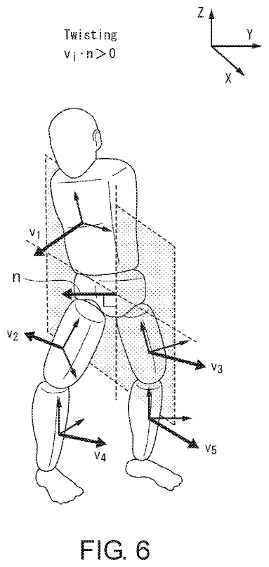

FIG. 6 is a diagram showing a state in which the direction vector vi is swiveled due to a change in the posture of the estimation target TGT. In the case where the output of the IMU sensor 40-p at a certain basis site is set as the initial state, the representative plane is swiveled in the yaw direction along the displacement in the yaw direction obtained by integrating the output of the IMU sensor 40-p. The correction part 160 increases the degree of correcting the converted angular velocity of the reference site as the orientation of the reference site calculated by the integration part 150 in the previous cycle continues to deviate from the orientation orthogonal to the normal line n of the sagittal plane.

[Posture Estimation]

For example, in the case where the inner product of the direction vector vi of the reference site and the normal line n is 0 as shown in FIG. 5, the correction part 160 determines that it is the posture of the home position in which the orientation of the reference site does not deviate from the orientation orthogonal to the normal line n of the sagittal plane, and in the case where the inner product of the direction vector vi and the normal line n is greater than 0 as shown in FIG. 6, the correction part 160 determines that the orientation of the reference site deviates from the orientation orthogonal to the normal line n of the sagittal plane. The home position is the basic posture (however, relative to the representative plane) of the estimation target TGT, which is obtained as a result of the first calibration process after the IMU sensors 40 are attached to the estimation target TGT, and is, for example, a stationary and upright state. The correction part 160 defines the home position based on the measurement results of the IMU sensors 40 obtained as a result of causing the estimation target TGT to perform a predetermined operation (calibration operation).

In this way, the correction part 160 makes corrections reflecting that the deviation decreases as time passes (approaching the home position as shown in FIG. 5) based on the assumption that it is rare that the estimation target maintains the posture deviated from the orientation orthogonal to the normal line n of the sagittal plane (that is, the state in which the body twists as shown in FIG. 6) for a long time, or moves while maintaining the posture deviated from the orientation orthogonal to the normal line n of the sagittal plane.

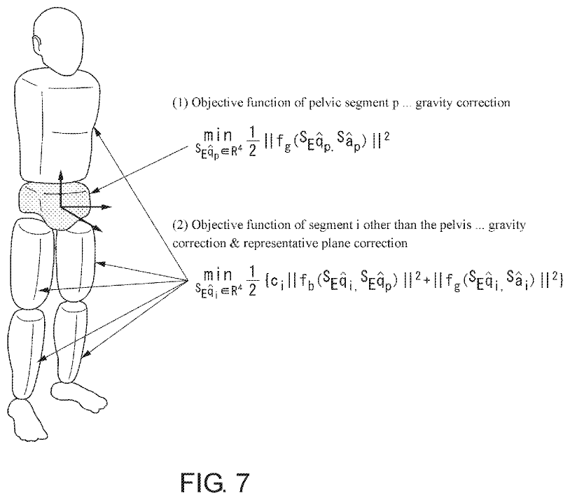

FIG. 7 is a diagram for schematically illustrating the correction process by the analysis device 100. The analysis device 100 defines an optimization problem which differs between the pelvis of the estimation target TGT and the other segments. First, the analysis device 100 calculates the pelvic posture of the estimation target TGT, and calculates postures of the other segments by using the pelvic posture.

Suppose that the calculation of the pelvic posture and the calculation of the postures of the other segments other than the pelvis are solved separately, then the pelvic posture ends up being estimated by using only gravity correction. The analysis device 100 simultaneously estimates the pelvic posture and the postures of the other segments so that the pelvic posture may be estimated in consideration of the postures of the other segments in order for optimization in consideration of the influence of all the IMU sensors 40.

### Calculation Example

Hereinafter, a specific calculation example at the time of estimating the posture will be described along with mathematical formulas.

An expression method of a quaternion for expressing a posture will be described. The rotation from a certain coordinate system frame A to frame B may be expressed by a quaternion as shown in the following formula (1). However, frame B is rotated by θ around the axis normalized to frame A.

$$\begin{matrix}[ Mathematical Formula 1 ] & \\ \begin{matrix}{}_{B}^{A} \hat{q} = {[ \begin{matrix}{q}_{1} & {q}_{2} \begin{matrix}{q}_{3} & {q}_{4}\end{matrix}\end{matrix} ]}^{T} = {[ cos \frac{θ}{2} - {r}_{x} sin \frac{θ}{2} - {r}_{y} sin \frac{θ}{2} - {r}_{z} sin \frac{θ}{2} ]}^{T} & \end{matrix} & ( 1 )\end{matrix}$$

Further, in the following description, a quaternion q with a hat symbol (a unit quaternion expressing rotation) will be described as “q(h)”. The unit quaternion is the quaternion divided by the norm. q(h) is a column vector having four real-valued elements as shown in the formula (1). When an estimated whole body posture vector Q of the estimation target TGT is expressed by using this expression method, it may be expressed as the following formula (2).

$$[ Mathematical Formula 2 ]$$

$$Q = [ \begin{matrix}{}_{E}^{S}{\hat{q}}{}_{p}^{} \\ {}_{E}^{S}{\hat{q}}{}_{1}^{} \\ {}_{E}^{S}{\hat{q}}{}_{2}^{} \\ ⋮ \\ {}_{E}^{S}{\hat{q}}{}_{i}^{} \\ ⋮ \\ {}_{E}^{S}{\hat{q}}{}_{N}^{}\end{matrix} ] ∈ {ℝ}^{4 ( N + 1 )} \qquad ( 2 )$$

In addition, ^{S}_{E}q(h)ᵢ (i is an integer of 1 to N indicating a segment or p indicating the basis position) expresses the rotation of the reference site from the basis position in the coordinate system S of the IMU sensors 40 (segment coordinate system) to a basis coordinate position E (for example, a coordinate system that may be defined from the gravity direction of the earth) in quaternions. The estimated whole body posture vector Q of the estimation target TGT is a column vector having 4 (N+1) real-valued elements that aggregates the unit quaternions expressing the postures of all the segments into one.

In order to estimate the posture of the estimation target TGT, first, the posture estimation of a certain segment to which the IMU sensor 40 is attached is considered.

$$[ Mathematical Formula 3 ]$$

$$\begin{matrix} {min}_{{}_{E}^{S} \hat{q} ∈ {ℝ}^{4}} \frac{1}{2} {‖ f ( {}_{E}^{S} \hat{q} , {}^{E} \hat{d} , {}^{S} \hat{s} ) ‖}^{2} & ( 3 ) \\ f ( {}_{E}^{S} \hat{q} , {}^{E} \hat{d} , {}^{S} \hat{s} ) = {}_{E}^{S}{\hat{q}}{}_{}^{*} ⊗ {}^{E} \hat{d} ⊗ {}_{E}^{S} \hat{q} - {}^{S} \hat{s} & ( 4 ) \\ {}_{E}^{S} \hat{q} = [ {q}_{1} {q}_{2} {q}_{3} {q}_{4} ] \text{:} Estimated IMU posture ( sensor coordinate system ) & ( 5 ) \\ {}^{E} \hat{d} = [ 0 {d}_{x} {d}_{y} {d}_{z} ] \text{:} Direction of basis such as gravity or geomagnetism ( constant \text{/} basis coordinate system ) & ( 6 ) \\ {}^{S} \hat{s} = [ 0 {s}_{x} {s}_{y} {s}_{z} ] \text{:} Measurement value of basis such as gravity or geomagnetism ( sensor coordinate system ) & ( 7 )\end{matrix}$$

The formula (3) is an example of an update formula of the optimization problem, and is a formula for deriving the correction amount in the roll and pitch directions by deriving the minimum value of ½ of the norm of the derivation result of the function shown in the formula (4). The right side of the formula (4) is a formula for subtracting the direction of the basis measured by the IMU sensor 40 expressed in the sensor coordinate system from the information indicating the direction in which the basis should be (for example, the direction of gravity or geomagnetism or the like) obtained from the estimated posture expressed in the sensor coordinate system.

As shown in the formula (5), ^{S}_{E}q is an example in which the unit quaternion ^{S}_{E}q(h) is expressed in a matrix form. Further, as shown in the formula (6), ^{E}d(h) is a vector indicating the direction of the basis (for example, the direction of gravity or geomagnetism or the like) used for correcting the yaw direction. Further, as shown in the formula (7), ^{S}s(h) is a vector indicating the direction of the basis measured by the IMU sensor 40 expressed in the sensor coordinate system.

In the case of using gravity as a basis, the formulas (6) and (7) may be expressed as the following formulas (8) and (9). aₓ, a_{y}, and a_{z} respectively indicate an acceleration in the x axis direction, an acceleration in the y axis direction, and an acceleration in the z axis direction.

^{E}d(h)=[0 0 0 1] (8)

^{S}s(h)=[0 aₓ a_{y} a_{z}] (9)

The relational expression shown in the formula (3) may be solved by, for example, the gradient descent method. In that case, the update formula of the estimated posture may be expressed by the formula (10). Further, the gradient of the objective function is expressed by the following formula (11). Further, the formula (11) indicating the gradient may be calculated by using the Jacobian as expressed by the formula (12). In addition, the Jacobian expressed by the formula (12) is a matrix obtained by partially differentiating the gravity error term and the yaw direction error term with each element of the direction vector vi of the whole body. The gravity error term and the yaw direction error term will be described later.

$$[ Mathematical Formula 4 ]$$

$$\begin{matrix}{}_{E}^{S}{\hat{q}}{}_{k + 1}^{} = {}_{E}^{S}{\hat{q}}{}_{k}^{} - μ ∇ \{ \frac{1}{2} {‖ f ( {}_{E}^{S} \hat{q} , {}^{E} \hat{d} , {}^{S} \hat{s} ) ‖}^{2} \} , k = 0 , 1 , 2 , … & ( 10 ) \\ ∇ \{ \frac{1}{2} {‖ f ( {}_{E}^{S} \hat{q} , {}^{E} \hat{d} , {}^{S} \hat{s} ) ‖}^{2} \} = {J}^{T} ( {}_{E}^{S}{\hat{q}}{}_{k}^{} , {}^{E} \hat{d} ) f ( {}_{E}^{S} \hat{q} , {}^{E} \hat{d} , {}^{S} \hat{s} ) & ( 11 ) \\ J ( {}_{E}^{S} \hat{q} , {}^{E} \hat{d} ) = [ \begin{matrix}\frac{\partial {f}_{1}}{\partial {q}_{1}} & \frac{\partial {f}_{1}}{\partial {q}_{2}} & \frac{\partial {f}_{1}}{\partial {q}_{3}} & \frac{\partial {f}_{1}}{\partial {q}_{4}} \\ \frac{\partial {f}_{2}}{\partial {q}_{1}} & \frac{\partial {f}_{2}}{\partial {q}_{2}} & \frac{\partial {f}_{2}}{\partial {q}_{3}} & \frac{\partial {f}_{2}}{\partial {q}_{4}} \\ \frac{\partial {f}_{3}}{\partial {q}_{1}} & \frac{\partial {f}_{3}}{\partial {q}_{2}} & \frac{\partial {f}_{3}}{\partial {q}_{3}} & \frac{\partial {f}_{3}}{\partial {q}_{4}}\end{matrix} ] & ( 12 )\end{matrix}$$

As shown on the right side of the formula (10), the unit quaternion ^{S}_{E}q(h)ₖ₊₁ may be derived by subtracting the product of the coefficient μ (constant less than or equal to 1) and the gradient from the unit quaternion ^{S}_{E}q(h)ₖ indicating the current estimated posture. Further, as shown in the formulas (11) and (12), the gradient may be derived with a relatively small amount of calculation.

The actual calculation examples of the formulas (4) and (12) in the case of using gravity as a basis are shown in the following formulas (13) and (14).

$$[ Mathematical Formula 5 ]$$

$$\begin{matrix}{f}_{g} ( {}_{E}^{S} \hat{q} , {}^{S} \hat{a} ) = [ \begin{matrix}2 ( {q}_{2} {q}_{4} - {q}_{1} {q}_{3} ) - {a}_{x} \\ 2 ( {q}_{1} {q}_{2} - {q}_{3} {q}_{4} ) - {a}_{y} \\ 2 ( \frac{1}{2} - {q}_{2}^{2} - {q}_{3}^{2} ) - {a}_{z}\end{matrix} ] & ( 13 ) \\ {J}_{g} ( {}_{E}^{S} \hat{q} ) = [ \begin{matrix}- 2 {q}_{3} & 2 {q}_{4} & - 2 {q}_{1} & 2 {q}_{2} \\ 2 {q}_{2} & 2 {q}_{1} & 2 {q}_{4} & 2 {q}_{3} \\ 0 & - 4 {q}_{2} & - 4 {q}_{3} & 0\end{matrix} ] & ( 14 )\end{matrix}$$

In the methods shown by using the formulas (3) to (7) and the formulas (10) to (12) in the above figure, the posture may be estimated by calculating the update formula once for each sampling. Further, in the case of using the gravity as a basis as exemplified in the formulas (8), (9), (13), and (14), corrections in the roll axis direction and the pitch axis direction may be performed.

[Whole Body Correction Amount Calculation]

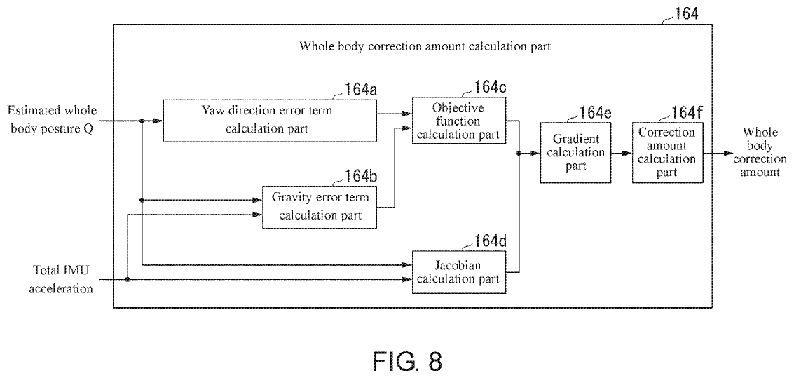

Hereinafter, a method for deriving the whole body correction amount (particularly the correction amount in the yaw direction) for the estimated posture will be described. FIG. 8 is a diagram showing an example of the configuration of the whole body correction amount calculation part 164. The whole body correction amount calculation part 164 includes, for example, a yaw direction error term calculation part 164a, a gravity error term calculation part 164b, an objective function calculation part 164c, a Jacobian calculation part 164d, a gradient calculation part 164e, and a correction amount calculation part 164f.

The yaw direction error term calculation part 164a calculates the yaw direction error term for realizing the correction in the yaw angle direction from the estimated whole body posture.

The gravity error term calculation part 164b calculates the gravity error term for realizing correction in the roll axis direction and the pitch axis direction from the estimated whole body posture and the acceleration detected by the IMU sensors 40.

The objective function calculation part 164c calculates an objective function for correcting the sagittal plane of the estimation target TGT and the direction vector vi to be parallel to each other based on the estimated whole body posture, the acceleration detected by the IMU sensors 40, the calculation result of the yaw direction error term calculation part 164a, and the calculation result of the gravity error term calculation part 164b. Further, the sum of squares of the gravity error term and the yaw direction error term is used as the objective function. The details of the objective function will be described later.

The Jacobian calculation part 164d calculates the Jacobian obtained by partial differentiation of the estimated whole body posture vector Q from the estimated whole body posture and the acceleration detected by the IMU sensors 40.

The gradient calculation part 164e derives a solution of the optimization problem by using the calculation result of the objective function calculation part 164c and the calculation result of the Jacobian calculation part 164d, and calculates the gradient.

The correction amount calculation part 164f derives the whole body correction amount to be applied to the estimated whole body posture vector Q of the estimation target TGT by using the calculation result of the gradient calculation part 164e.

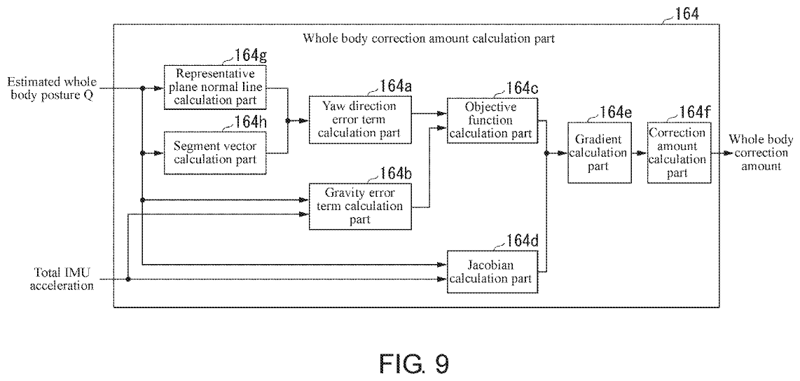

FIG. 9 is a diagram showing another example of the configuration of the whole body correction amount calculation part 164. The whole body correction amount calculation part 164 shown in FIG. 9 derives the whole body correction amount by using the sagittal plane and the direction vector vi of each segment, and in addition to the components shown in FIG. 8, further includes a representative plane normal line calculation part 164g and a segment vector calculation part 164h.

The representative plane normal line calculation part 164g calculates the normal line n of the sagittal plane, which is the representative plane, based on the estimated whole body posture. The segment vector calculation part 164h calculates the direction vector vi of the segment based on the estimated whole body posture.

[Example of Deriving Whole Body Correction Amount]

Hereinafter, an example of deriving the whole body correction amount will be described.

The yaw direction error term calculation part 164a calculates the inner product of the yaw direction error term f_{b} for correcting the sagittal plane and the direction vector of the segment to be parallel to each other by using the following formula (15).

[Mathematical Formula 6]

f_{b}(^{S}_{E}\hat{q}ᵢ,^{S}_{E}\hat{q}ₚ)=(^{S}_{E}\hat{q}ₚ⊗Sₙ⊗^{S}_{E}\hat{q}ₚ*)·(^{S}_{E}\hat{q}ᵢ⊗^{S}vᵢ⊗^{S}_{E}\hat{q}ᵢ*)∈ [symbol] (15)

The yaw direction error term f_{b} is a formula for deriving a correction amount based on the unit quaternion ^{S}_{E}q(h)ᵢ indicating the estimated posture of the segment i and the unit quaternion ^{S}_{E}q(h)ₚ indicating the estimated posture of the pelvis which is the basis site. The right side of the formula (15) derives the inner product of the normal line n of the sagittal plane, which is expressed in the sensor coordinate system and calculated by the representative plane normal line calculation part 164g, and the direction vector vi of the segment, which is expressed in the sensor coordinate system and calculated by the segment vector calculation part 164h. In this way, in the case where the body of the estimation target TGT is in a twisting state, the correction may be performed with the correction content in which the twist is eliminated (approaching the home position as shown in FIG. 5).

Next, the gravity error term calculation part 164b performs a calculation for performing basis correction (for example, gravity correction) for each segment as shown in the formula (16).

[Mathematical Formula 7]

f_{g}(^{S}_{E}\hat{q}ᵢ,^{S}âᵢ)=^{S}_{E}\hat{q}ᵢ*⊗^{E}\hat{d}_{g}⊗^{S}_{E}\hat{q}ᵢ−^{S}âᵢ (16)

The formula (16) is a relational formula between the unit quaternion ^{S}_{E}q(h)ᵢ indicating the estimated posture of any segment i and the acceleration (gravity) measured by the IMU sensor 40-i. As shown on the right side of the formula (16), it may be derived by subtracting the measured gravity direction (measured gravitational acceleration direction) ^{S}aᵢ(h) expressed in the sensor coordinate system from the direction in which gravity should be (assumed gravitational acceleration direction) expressed in the sensor coordinate system obtained from the estimated posture.

Here, a specific example of the measured gravity direction ^{S}aᵢ(h) is shown in the formula (17). Further, the constant ^{E}d_{g}(h) indicating the gravity direction may be expressed by a constant as shown in the formula (18).

[Mathematical Formula 8]

^{S}âᵢ=[0 a_{i,x} a_{i,v} a_{i,z}] (17)

^{E}\hat{d}_{g}=[0 0 0 1]^{T} (18)

Next, the objective function calculation part 164c calculates the formula (19) as the correction function of the segment i, which integrates the gravity error term and the yaw direction error term.

$$[ Mathematical Formula 9 ]$$

$${f}_{i} ( {}_{E}^{S}{\hat{q}}{}_{i}^{} , {}_{E}^{S}{\hat{q}}{}_{p}^{} , {}_{}^{S}{\hat{a}}{}_{i}^{} ) = [ \begin{matrix}\sqrt{{c}_{i}} {f}_{b} ( {}_{E}^{S}{\hat{q}}{}_{i}^{} , {}_{E}^{S}{\hat{q}}{}_{p}^{} ) \\ {f}_{g} ( {}_{E}^{S}{\hat{q}}{}_{i}^{} , {}_{}^{S}{\hat{a}}{}_{i}^{} ) \end{matrix} ] ∈ {ℝ}^{4} \qquad ( 19 )$$

Here, cᵢ is a weighting coefficient for representative plane correction. The formula (19) showing the correction function of the segment i may be expressed as the formula (20) when formalized as an optimization problem.

$$[ Mathematical Formula 10 ]$$

$${min}_{{}_{E}^{S}{\hat{q}}{}_{i}^{} ∈ {ℝ}^{4}} \frac{1}{2} {‖ {f}_{i} ( {}_{E}^{S}{\hat{q}}{}_{i}^{} , {}_{E}^{S}{\hat{q}}{}_{p}^{} , {}_{}^{S}{\hat{a}}{}_{i}^{} ) ‖}^{2} \qquad ( 20 )$$

Further, the formula (20) is equivalent to the formula (21) of the correction function which may be expressed by the sum of the objective functions of the gravity correction and the representative plane correction.

$$[ Mathematical Formula 11 ]$$

$${min}_{{}_{E}^{S}{\hat{q}}{}_{i}^{} , ∈ {ℝ}^{4}} \frac{1}{2} \{ {c}_{i} {‖ {f}_{b} ( {}_{E}^{S}{\hat{q}}{}_{i}^{} , {}_{E}^{S}{\hat{q}}{}_{p}^{} ) ‖}^{2} + {‖ {f}_{g} ( {}_{E}^{S}{\hat{q}}{}_{i}^{} , {}_{}^{S}{\hat{a}}{}_{i}^{} ) ‖}^{2} \} \qquad ( 21 )$$

The objective function calculation part 164c performs posture estimation for all segments in the same manner, and defines an optimization problem which integrates the objective functions of the whole body. The formula (22) is a correction function F(Q, α) which integrates the objective functions of the whole body. α is the total IMU acceleration measured by the IMU sensor and may be expressed as in the formula (23).

$$[ Mathematical Formula 12 ]$$

$$\begin{matrix}F ( Q , α ) = [ \begin{matrix}{f}_{p} ( {}_{E}^{S}{\hat{q}}{}_{p}^{} , {}_{}^{S}{\hat{a}}{}_{p}^{} ) \\ {f}_{1} ( {}_{E}^{S}{\hat{q}}{}_{1}^{} , {}_{E}^{S}{\hat{q}}{}_{p}^{} , {}_{}^{S}{\hat{a}}{}_{1}^{} ) \\ {f}_{2} ( {}_{E}^{S}{\hat{q}}{}_{2}^{} , {}_{E}^{S}{\hat{q}}{}_{p}^{} , {}_{}^{S}{\hat{a}}{}_{2}^{} ) \\ ⋮ \\ {f}_{i} ( {}_{E}^{S}{\hat{q}}{}_{i}^{} , {}_{E}^{S}{\hat{q}}{}_{p}^{} , {}_{}^{S}{\hat{a}}{}_{i}^{} ) \\ ⋮ \\ {f}_{N} ( {}_{E}^{S}{\hat{q}}{}_{N}^{} , {}_{E}^{S}{\hat{q}}{}_{p}^{} , {}_{}^{S}{\hat{a}}{}_{N}^{} )\end{matrix} ] ∈ {ℝ}^{( 3 + 4 N )} & ( 22 ) \\ α = [ \begin{matrix}{}_{}^{S}{\hat{a}}{}_{p}^{} \\ {}_{}^{S}{\hat{a}}{}_{1}^{} \\ {}_{}^{S}{\hat{a}}{}_{2}^{} \\ ⋮ \\ {}_{}^{S}{\hat{a}}{}_{i}^{} \\ ⋮ \\ {}_{}^{S}{\hat{a}}{}_{N}^{}\end{matrix} ] ∈ {ℝ}^{4 ( N + 1 )} & ( 23 )\end{matrix}$$

Further, the first line on the right side of the formula (22) expresses the correction function corresponding to the pelvis, and the second and subsequent lines on the right side express the correction function corresponding to each segment other than the pelvis. By using the correction functions expressed in the formula (22), the optimization problem for correcting the posture of the whole body of the estimation target TGT may be defined as in the formula (24) below. The formula (24) may be modified as expressed in the formula (25) in the same form as the formula (21) which is the correction function of each segment already described.

$$[ Mathematical Formula 13 ]$$

$$\begin{matrix}{min}_{Q ∈ {ℝ}^{4 ( N + 1 )}} \frac{1}{2} {‖ F ( Q , α ) ‖}^{2} & ( 24 ) \\ {min}_{Q ∈ {ℝ}^{4 ( N + 1 )}} \frac{1}{2} \{ {‖ {f}_{p} ( {}_{E}^{S}{\hat{q}}{}_{p}^{} , {}_{}^{S}{\hat{a}}{}_{p}^{} ) ‖}^{2} + {\sum }_{i = 1}^{N} {‖ {f}_{i} ( {}_{E}^{S}{\hat{q}}{}_{i}^{} , {}_{E}^{S}{\hat{q}}{}_{p}^{} , {}_{}^{S}{\hat{a}}{}_{i}^{} ) ‖}^{2} \} & ( 25 )\end{matrix}$$

Next, the gradient calculation part 164e calculates the gradient of this objective function as expressed in the following formula (26) by using the Jacobian J_{F} obtained by the partial differentiation of the estimated whole body posture vector Q. Further, the Jacobian J_{F} is expressed in the formula (27).

$$[ Mathematical Formula 14 ]$$

$$\begin{matrix}\frac{1}{2} ∇ {‖ F ( Q , α ) ‖}^{2} = {J}_{F}^{T} ( Q , α ) F ( Q , α ) & ( 26 ) \\ {J}_{F} ( Q , α ) = [ \begin{matrix}\frac{\partial {f}_{p} ( {}_{E}^{S}{\hat{q}}{}_{p}^{} , {}_{}^{S}{\hat{a}}{}_{p}^{} )}{\partial {}_{E}^{S}{\hat{q}}{}_{p}^{}} & \frac{\partial {f}_{p} ( {}_{E}^{S}{\hat{q}}{}_{p}^{} , {}_{}^{S}{\hat{a}}{}_{p}^{} )}{\partial {}_{E}^{S}{\hat{q}}{}_{1}^{}} & ⋯ & \frac{\partial {f}_{p} ( {}_{E}^{S}{\hat{q}}{}_{p}^{} , {}_{}^{S}{\hat{a}}{}_{p}^{} )}{\partial {}_{E}^{S}{\hat{q}}{}_{i}^{}} & ⋯ & \frac{\partial {f}_{p} ( {}_{E}^{S}{\hat{q}}{}_{p}^{} , {}_{}^{S}{\hat{a}}{}_{p}^{} )}{\partial {}_{E}^{S}{\hat{q}}{}_{N}^{}} \\ \frac{\partial {f}_{1} ( {}_{E}^{S}{\hat{q}}{}_{1}^{} , {}_{E}^{S}{\hat{q}}{}_{p}^{} , {}_{}^{S}{\hat{a}}{}_{1}^{} )}{\partial {}_{E}^{S}{\hat{q}}{}_{p}^{}} & \frac{\partial {f}_{1} ( {}_{E}^{S}{\hat{q}}{}_{1}^{} , {}_{E}^{S}{\hat{q}}{}_{p}^{} , {}_{}^{S}{\hat{a}}{}_{1}^{} )}{\partial {}_{E}^{S}{\hat{q}}{}_{1}^{}} & ⋯ & \frac{\partial {f}_{1} ( {}_{E}^{S}{\hat{q}}{}_{1}^{} , {}_{E}^{S}{\hat{q}}{}_{p}^{} , {}_{}^{S}{\hat{a}}{}_{1}^{} )}{\partial {}_{E}^{S}{\hat{q}}{}_{i}^{}} & ⋯ & \frac{\partial {f}_{1} ( {}_{E}^{S}{\hat{q}}{}_{1}^{} , {}_{E}^{S}{\hat{q}}{}_{p}^{} , {}_{}^{S}{\hat{a}}{}_{1}^{} )}{\partial {}_{E}^{S}{\hat{q}}{}_{N}^{}} \\ ⋮ & ⋮ & ⋱ & ⋮ & ⋱ & ⋮ \\ \frac{\partial {f}_{i} ( {}_{E}^{S}{\hat{q}}{}_{i}^{} , {}_{E}^{S}{\hat{q}}{}_{p}^{} , {}_{}^{S}{\hat{a}}{}_{i}^{} )}{\partial {}_{E}^{S}{\hat{q}}{}_{p}^{}} & \frac{\partial {f}_{i} ( {}_{E}^{S}{\hat{q}}{}_{i}^{} , {}_{E}^{S}{\hat{q}}{}_{p}^{} , {}_{}^{S}{\hat{a}}{}_{i}^{} )}{\partial {}_{E}^{S}{\hat{q}}{}_{1}^{}} & ⋯ & \frac{\partial {f}_{i} ( {}_{E}^{S}{\hat{q}}{}_{i}^{} , {}_{E}^{S}{\hat{q}}{}_{p}^{} , {}_{}^{S}{\hat{a}}{}_{i}^{} )}{\partial {}_{E}^{S}{\hat{q}}{}_{i}^{}} & ⋯ & \frac{\partial {f}_{i} ( {}_{E}^{S}{\hat{q}}{}_{i}^{} , {}_{E}^{S}{\hat{q}}{}_{p}^{} , {}_{}^{S}{\hat{a}}{}_{i}^{} )}{\partial {}_{E}^{S}{\hat{q}}{}_{N}^{}} \\ ⋮ & ⋮ & ⋱ & ⋮ & ⋱ & ⋮ \\ \frac{\partial {f}_{N} ( {}_{E}^{S}{\hat{q}}{}_{N}^{} , {}_{E}^{S}{\hat{q}}{}_{p}^{} , {}_{}^{S}{\hat{a}}{}_{N}^{} )}{\partial {}_{E}^{S}{\hat{q}}{}_{p}^{}} & \frac{\partial {f}_{N} ( {}_{E}^{S}{\hat{q}}{}_{N}^{} , {}_{E}^{S}{\hat{q}}{}_{p}^{} , {}_{}^{S}{\hat{a}}{}_{N}^{} )}{\partial {}_{E}^{S}{\hat{q}}{}_{1}^{}} & ⋯ & \frac{\partial {f}_{N} ( {}_{E}^{S}{\hat{q}}{}_{N}^{} , {}_{E}^{S}{\hat{q}}{}_{p}^{} , {}_{}^{S}{\hat{a}}{}_{N}^{} )}{\partial {}_{E}^{S}{\hat{q}}{}_{i}^{}} & ⋯ & \frac{\partial {f}_{N} ( {}_{E}^{S}{\hat{q}}{}_{N}^{} , {}_{E}^{S}{\hat{q}}{}_{p}^{} , {}_{}^{S}{\hat{a}}{}_{N}^{} )}{\partial {}_{E}^{S}{\hat{q}}{}_{N}^{}}\end{matrix} ] ∈ {ℝ}^{( 3 + 4 N ) \times 4 ( N + 1 )} & ( 27 )\end{matrix}$$

The size of each element expressed in the formula (27) is as expressed in the following formulas (28) and (29).

$$[ Mathematical Formula 15 ]$$

$$\begin{matrix}\frac{\partial {f}_{p} ( {}_{E}^{S}{\hat{q}}{}_{p}^{} , {}_{}^{S}{\hat{a}}{}_{p}^{} )}{\partial {}_{E}^{S}{\hat{q}}{}_{p}^{}} , \frac{\partial {f}_{p} ( {}_{E}^{S}{\hat{q}}{}_{p}^{} , {}_{}^{S}{\hat{a}}{}_{p}^{} )}{\partial {}_{E}^{S}{\hat{q}}{}_{i}^{}} ∈ {ℝ}^{3 \times 4} & ( 28 ) \\ \frac{\partial {f}_{i} ( {}_{E}^{S}{\hat{q}}{}_{i}^{} , {}_{E}^{S}{\hat{q}}{}_{p}^{} , {}_{}^{S}{\hat{a}}{}_{i}^{} )}{\partial {}_{E}^{S}{\hat{q}}{}_{p}^{}} , \frac{\partial {f}_{i} ( {}_{E}^{S}{\hat{q}}{}_{i}^{} , {}_{E}^{S}{\hat{q}}{}_{p}^{} , {}_{}^{S}{\hat{a}}{}_{i}^{} )}{\partial {}_{E}^{S}{\hat{q}}{}_{i}^{}} ∈ {ℝ}^{4 \times 4} & ( 29 )\end{matrix}$$

That is, the Jacobian J_{F} expressed in the formula (27) is a large matrix of (3+4N)×4 (N+1) (N is the total number of the IMU sensors other than the IMU sensor for measuring the basis site), but in reality, since the elements expressed in the following formulas (30) and (31) are 0, the calculation may be omitted, and real-time posture estimation is possible even with a low-speed arithmetic device.

$$[ Mathematical Formula 16 ]$$

$$\begin{matrix}\frac{\partial {f}_{p} ( {}_{E}^{S}{\hat{q}}{}_{p}^{} , {}_{}^{S}{\hat{a}}{}_{p}^{} )}{\partial {}_{E}^{S}{\hat{q}}{}_{i}^{}} = 0 , {∀}_{i} ∈ [ 1 , N ] & ( 30 ) \\ \frac{\partial {f}_{i} ( {}_{E}^{S}{\hat{q}}{}_{i}^{} , {}_{E}^{S}{\hat{q}}{}_{p}^{} , {}_{}^{S}{\hat{a}}{}_{i}^{} )}{\partial {}_{E}^{S}{\hat{q}}{}_{j}^{}} = 0 , i \neq j & ( 31 )\end{matrix}$$

Substituting the formulas (30) and (31) into the above formula (27), it may be expressed as the following formula (32).

$$[ Mathematical Formula 17 ]$$

$${J}_{F} ( Q , α ) = [ \begin{matrix}\frac{\partial {f}_{p} ( {}_{E}^{S}{\hat{q}}{}_{p}^{} , {}_{}^{S}{\hat{a}}{}_{p}^{} )}{\partial {}_{E}^{S}{\hat{q}}{}_{p}^{}} & 0 & ⋯ & 0 & ⋯ & 0 \\ \frac{\partial {f}_{1} ( {}_{E}^{S}{\hat{q}}{}_{1}^{} , {}_{E}^{S}{\hat{q}}{}_{p}^{} , {}_{}^{S}{\hat{a}}{}_{1}^{} )}{\partial {}_{E}^{S}{\hat{q}}{}_{p}^{}} & \frac{\partial {f}_{1} ( {}_{E}^{S}{\hat{q}}{}_{1}^{} , {}_{E}^{S}{\hat{q}}{}_{p}^{} , {}_{}^{S}{\hat{a}}{}_{1}^{} )}{\partial {}_{E}^{S}{\hat{q}}{}_{1}^{}} & ⋯ & 0 & ⋯ & 0 \\ ⋮ & ⋮ & ⋱ & ⋮ & ⋱ & ⋮ \\ \frac{\partial {f}_{i} ( {}_{E}^{S}{\hat{q}}{}_{i}^{} , {}_{E}^{S}{\hat{q}}{}_{p}^{} , {}_{}^{S}{\hat{a}}{}_{i}^{} )}{\partial {}_{E}^{S}{\hat{q}}{}_{p}^{}} & 0 & ⋯ & \frac{\partial {f}_{i} ( {}_{E}^{S}{\hat{q}}{}_{i}^{} , {}_{E}^{S}{\hat{q}}{}_{p}^{} , {}_{}^{S}{\hat{a}}{}_{i}^{} )}{\partial {}_{E}^{S}{\hat{q}}{}_{i}^{}} & ⋯ & 0 \\ ⋮ & ⋮ & ⋱ & ⋮ & ⋱ & ⋮ \\ \frac{\partial {f}_{N} ( {}_{E}^{S}{\hat{q}}{}_{N}^{} , {}_{E}^{S}{\hat{q}}{}_{p}^{} , {}_{}^{S}{\hat{a}}{}_{N}^{} )}{\partial {}_{E}^{S}{\hat{q}}{}_{p}^{}} & 0 & ⋯ & 0 & ⋯ & \frac{\partial {f}_{N} ( {}_{E}^{S}{\hat{q}}{}_{N}^{} , {}_{E}^{S}{\hat{q}}{}_{p}^{} , {}_{}^{S}{\hat{a}}{}_{N}^{} )}{\partial {}_{E}^{S}{\hat{q}}{}_{N}^{}}\end{matrix} ] ∈ {ℝ}^{( 3 + 4 N ) \times 4 ( N + 1 )} \qquad ( 32 )$$

The gradient calculation part 164e may calculate the gradient expressed in the formula (26) by using the calculation result of the formula (32).

[Process Image of Whole Body Correction Amount Calculation Part]

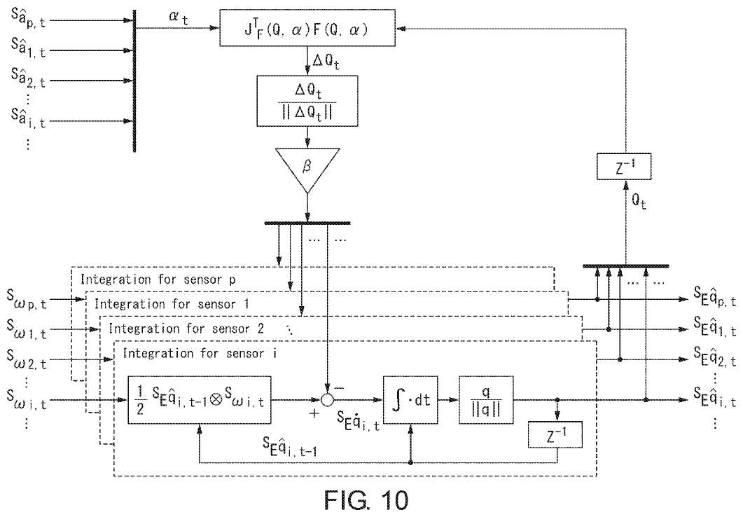

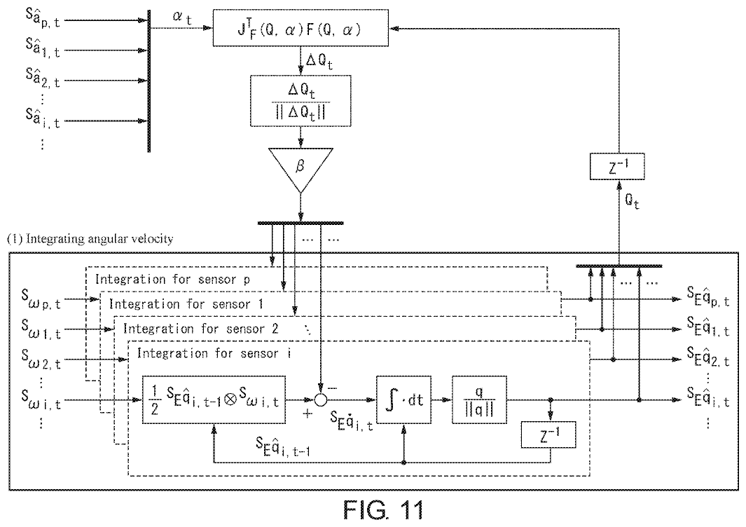

FIGS. 10 to 13 are diagrams schematically showing the flow of the arithmetic process of the whole body correction amount calculation part 164. FIG. 10 is a diagram schematically showing the overall process of the whole body correction amount calculation part 164, and FIGS. 11 to 13 are diagrams for stepwise illustrating the flow of the process of the whole body correction amount calculation part 164.

As shown in FIG. 10, the acceleration aggregation part 148 converts the obtaining result of the first obtaining part 130 of the acceleration ^{S}a_{i, t} of each IMU sensor 40-i (i may be p indicating the pelvis as the basis site, and the same applies hereinafter) measured at time t, and converts it into the total IMU acceleration aₜ of the estimation target TGT which is the aggregation result. Further, the angular velocity ^{S}ω_{i, t} of each IMU sensor 40-i measured at time t obtained by the first obtaining part 130 is output to the corresponding angular velocity integration part 152-i.

Further, the process blocks from Z⁻¹ to β shown in the upper right part of FIG. 10 represent that the correction part 160 derives the correction amount in the next process cycle.

Further, in FIGS. 10 to 13, assuming that the gradient of the objective function expressed by the following formula (33) is ΔQₜ, the feedback to the angular velocity Qₜ(⋅) (the dot symbol is added as the upper character of Qₜ, indicating the time derivative result of the estimated whole body posture vector Qₜ at time t) at time t may be expressed by the following formula (34). Further, β in the formula (34) is a real number in which 0≤β≤1 for adjusting the gain of the correction amount.

$$[ Mathematical Formula 18 ]$$

$$\begin{matrix}Δ Q = {J}_{F}^{T} ( Q , α ) F ( Q , α ) & ( 33 ) \\ {\dot{Q}}_{t} ← {\dot{Q}}_{t} - β \frac{Δ {Q}_{t}}{‖ Δ {Q}_{t} ‖} & ( 34 )\end{matrix}$$

As shown in the formula (34), the whole body correction amount calculation part 164 reflects an arbitrary real number β as a correction amount in the result of normalizing the gradient ΔQ to the angular velocity Qₜ(⋅).

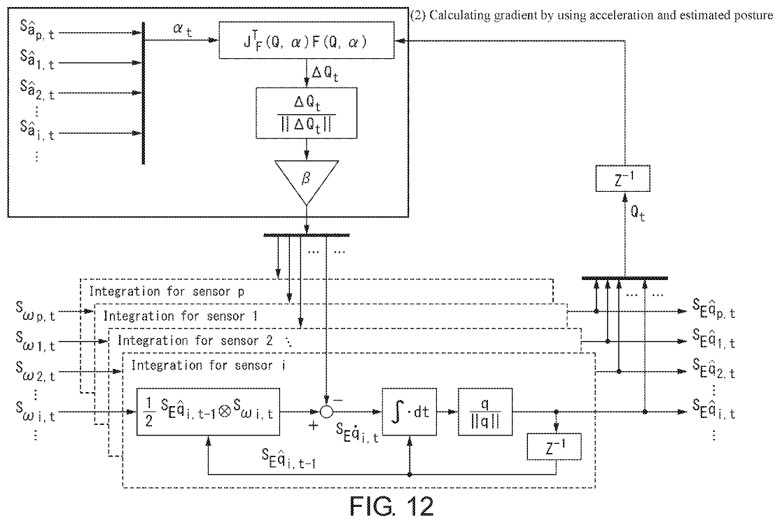

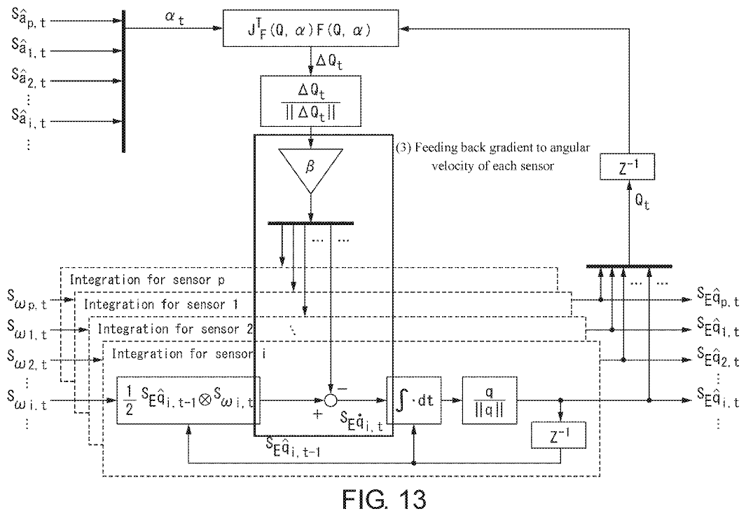

As shown in FIG. 11, the integration part 150 integrates the angular velocity of each segment. Next, as shown in FIG. 12, the correction part 160 calculates the gradient ΔQ by using the angular velocity and the estimated posture of each segment. Next, as shown in FIG. 13, the correction part 160 feeds back the derived gradient ΔQ to the angular velocity of each IMU sensor. When the first obtaining part 130 obtains the next measurement result by the IMU sensors 40, the integration part 150 integrates the angular velocity of each segment again as shown in FIG. 11. The analysis device 100 performs the posture estimation process of the estimation target TGT by repeating the processes shown in FIGS. 11 to 13, and since the characteristics and empirical rules of the human body are reflected in the posture estimation result of each segment, the accuracy of the estimation result of the analysis device 100 is improved.

The processes shown in FIGS. 11 to 13 are repeatedly performed, and the estimated posture aggregation part 162 aggregates the integration results of the angular velocities of the integration part 150, whereby the errors of the measured angular velocities of each of the IMU sensors 40 are averaged, and the estimated whole body posture vector Q of the formula (2) may be derived. This estimated whole body posture vector Q reflects the result of calculating the yaw direction correction amount from the whole body posture by using the characteristics and empirical rules of the human body. By performing the posture estimation of the estimation target TGT by the above method, it is possible to estimate the whole body posture of a plausible person while suppressing the yaw angle direction drift without using geomagnetism, so even in the case of performing measurement for a long time, it is possible to estimate the whole body posture while suppressing the yaw direction drift.

The analysis device 100 stores the whole body posture estimation result in the storage part 190 as the analysis result, and provides the terminal device 10 with information indicating the analysis result.

[Calibration Process]

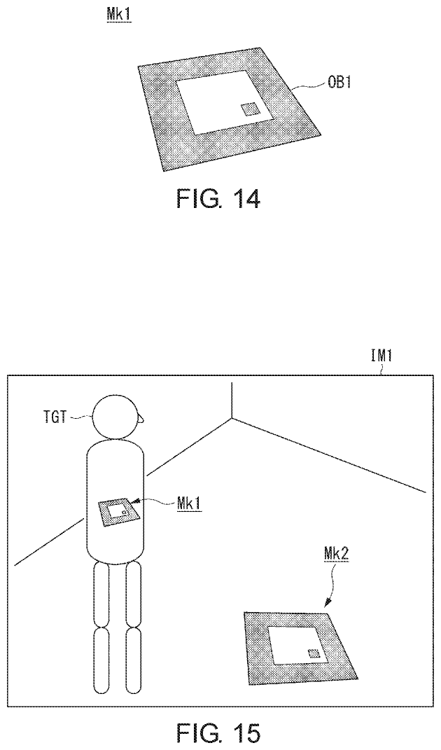

Hereinafter, an example of the calibration process by the calibration part 180 will be described. The second obtaining part 170 obtains an image (hereinafter referred to as a captured image) captured by the image capturing part of the image capturing device 50. The image capturing device 50 is flight-controlled to capture an image of the estimation target TGT, for example, by control from the terminal device 10 (which may be an automatic control or a manual control). One or more first markers are provided on the estimation target TGT. The first marker may be printed on the measurement wear 30, or may be attached as a sticker. The first marker includes an image that may be easily recognized by a machine, and its position and posture change in conjunction with the segment of the provided position. It is preferable that the image includes an image showing a spatial direction. FIG. 14 is a diagram showing an example of the appearance of the first marker Mk1. For example, the first marker Mk1 is drawn with a contrast that may be easily extracted from the captured image, and has a two-dimensional shape such as a rectangle.

FIG. 15 is a diagram showing an example of a captured image IM1. The image capturing device 50 is controlled so that the captured image IM1 includes a second marker Mk2 in addition to the first marker Mk1. The second marker Mk2 is provided on a stationary body such as a floor surface. Like the first marker Mk1, the second marker Mk2 is also drawn with a contrast that may be easily extracted from the captured image, and has a two-dimensional shape such as a rectangle.

It is assumed that the posture of the first marker Mk1 matches the sensor coordinate system. The first marker Mk1 is provided, for example, in such a manner that the posture relative to the posture of the IMU sensor 40 does not change. For example, the first marker Mk1 is printed or attached to a rigid body member which configures the IMU sensor 40. The calibration part 180 calibrates the conversion rule from the sensor coordinate system to the segment coordinate system based on the first marker Mk1 and the second marker Mk2 in the captured image IM. The “conversion part” in the claims includes at least the primary conversion part 140, and may further include the integration part 150 and the correction part 160. Therefore, the conversion rule may refer to a rule by which the primary conversion part 140 converts the angular velocity of the IMU sensor 40-i into information of the segment coordinate system, and may further refer to a rule including processes performed by the integration part 150 and the correction part 160.

Here, the sensor coordinate system is defined as <M>; the segment coordinate system is defined as <S>; the camera coordinate system whose origin is the position of the image capturing device 50 is defined as <E>; and the global coordinate system which is a stationary coordinate system is defined as <G>. The global coordinate system <G> is, for example, a ground coordinate system with the gravity direction as one axis. The calibration target is the conversion rule (hereinafter, conversion matrix) _{M}^{S}R from the sensor coordinate system <M> to the segment coordinate system <S>.

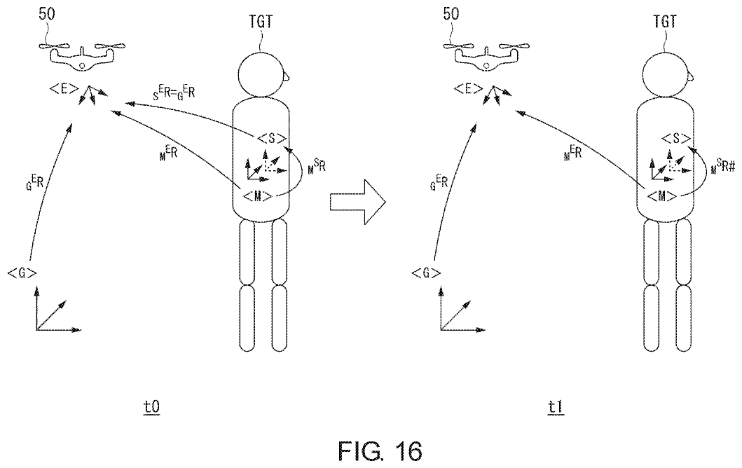

FIG. 16 is a diagram for illustrating the content of the process by the calibration part 180. At the home position setting time t0 described above, the calibration part 180 obtains the captured image IM as shown in FIG. 15, derives the posture of the first marker Mk1 with respect to the image capturing part based on the position of the apex of the first marker Mk1, and obtains the rotation angle between the coordinate systems from the derived posture, thereby deriving the conversion matrix _{M}^{E}R from the sensor coordinate system <M> to the camera coordinate system <E>. Relevant techniques are known, for example, as a function of OpenCV. Further, the calibration part 180 derives the posture of the second marker Mk2 with respect to the image capturing part based on the position of the apex of the second marker Mk2 and obtains the rotation angle between the coordinate systems from the derived posture, thereby deriving the conversion matrix _{G}^{E}R from the global coordinate system <G> to the camera coordinate system <E>. At this time, in the case where the estimation target TGT is in an upright posture, it may be assumed that the segment coordinate system <S> and the global coordinate system <G> match. Therefore, it may be assumed that the conversion matrix _{S}^{E}R=the conversion matrix _{G}^{E}R. At this time, the conversion matrix from the sensor coordinate system <M> to the segment coordinate system <S> is defined as _{M}^{S}R.

When the position and posture of the IMU sensor 40 with respect to the estimation target TGT shifts at the calibration time t1 after the home position setting time t0, the conversion matrix from the sensor coordinate system <M> to the segment coordinate system <S> changes to _{M}^{S}R#. At this time, the conversion matrix _{M}^{S}R# is obtained by the formula (35). Since it may be assumed that _{S}^{E}R=_{G}^{E}R as described above, the relationship of the formula (36) may be obtained in the case where the estimation target TGT takes the same upright posture as the home position setting time t0. Therefore, by multiplying the inverse matrix _{E}^{G}R of the conversion matrix _{G}^{E}R from the global coordinate system <G> to the camera coordinate system <E> and the conversion matrix _{M}^{E}R from the sensor coordinate system <M> to the camera coordinate system <E>, the conversion matrix _{M}^{S}R# from the sensor coordinate system <M> to the segment coordinate system <S> may be derived.

$$\begin{matrix}{}_{M}^{S} R \# = {}_{S}^{E}{R}{}_{}^{T} \cdot {}_{M}^{E} R & ( 35 ) \\ \begin{matrix}{}_{M}^{S} R \# = {}_{G}^{E}{R}{}_{}^{T} \cdot {}_{M}^{E} R \\ = {( {}_{E}^{G}{R}{}_{}^{T} )}^{T} \cdot {}_{M}^{E} R \\ = {}_{E}^{G} R \cdot {}_{M}^{E} R\end{matrix} & ( 36 )\end{matrix}$$

When the conversion matrix _{M}^{S}R# from the sensor coordinate system <M> to the segment coordinate system <S> is obtained as described above, the calibration part 180 calibrates the conversion rule from the sensor coordinate system to the segment coordinate system based on the conversion matrix _{M}^{S}R#. Thereby, at the calibration time t1 after the home position setting time t0, the calibration related to the posture estimation by using the IMU sensor 40 may be appropriately performed.

According to the first embodiment described above, calibration related to the posture estimation by using the IMU sensor 40 may be appropriately performed.

### Second Embodiment

Hereinafter, a second embodiment will be described. The second embodiment is different from the first embodiment in that the process content of the calibration part 180 is different. Therefore, the differences will be mainly described.

In the second embodiment, one or more third markers Mk3 are provided on the estimation target TGT. Unlike the first marker Mk1, the third marker Mk3 shows an axis figure indicating the axial direction of the segment coordinate system. Further, in the second embodiment, the second marker Mk2 is not a required configuration, but its presence may be expected to improve the accuracy.

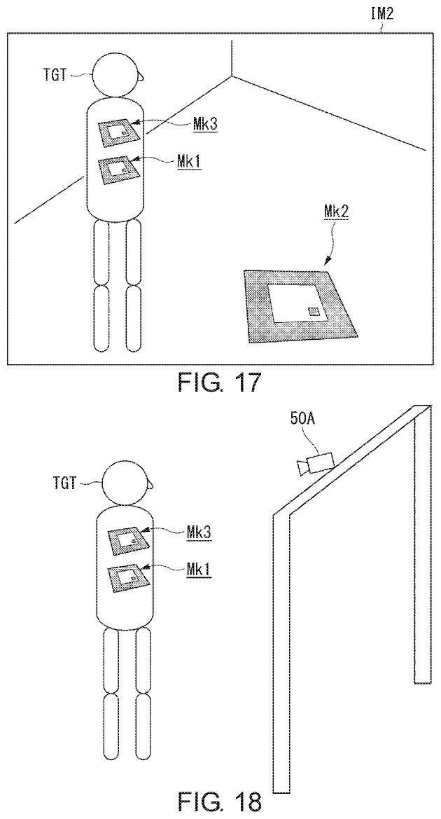

FIG. 17 is a diagram showing an example of a captured image IM2. The image capturing device 50 is controlled so that the captured image IM2 includes the third marker Mk3 in addition to the first marker Mk1. In the example of FIG. 17, the second marker Mk2 is captured. For example, the third marker Mk3 is drawn with a contrast that may be easily extracted from the captured image, and has a two-dimensional shape such as a rectangle.

It is assumed that the posture of the third marker Mk3 matches the segment coordinate system. For example, the third marker Mk3 is printed or attached to the measurement wear 30 to contact a site of the estimation target TGT close to a rigid body such as the pelvis or the spine. The calibration part 180 calibrates the conversion rule from the sensor coordinate system to the segment coordinate system based on the first marker Mk1 and the axis figure of the third marker Mk3 in the captured image IM.

The description will be given according to the same definition as in the first embodiment. At the home position setting time t0 described above and the calibration time t1 thereafter, the calibration part 180 obtains the captured image IM as shown in FIG. 17 and derives the conversion matrix _{M}^{E}R from the sensor coordinate system <M> to the camera coordinate system <E> based on the position of the apex of the first marker Mk1. Further, the calibration part 180 derives the posture of the third marker Mk3 with respect to the image capturing part based on the position of the apex of the third marker Mk3 and obtains the rotation angle between the coordinate systems from the derived posture, thereby deriving the conversion matrix _{S}^{E}R from the segment coordinate system <S> to the camera coordinate system <E>. The conversion matrix _{M}^{S}R# from the sensor coordinate system <M> to the segment coordinate system <S> at the calibration time t1 is directly obtained by the above formula (35).

When the conversion matrix _{M}^{S}R# from the sensor coordinate system <M> to the segment coordinate system <S> is obtained as described above, the calibration part 180 calibrates the conversion rule from the sensor coordinate system to the segment coordinate system based on the conversion matrix _{M}^{S}R#. Thereby, at the calibration time t1 after the home position setting time t0, the calibration related to the posture estimation by using the IMU sensor 40 may be appropriately performed.

According to the second embodiment described above, calibration related to the posture estimation by using the IMU sensor 40 may be appropriately performed.

<Modified Example of the Second Embodiment>

In the second embodiment, the calibration part 180 derives the conversion matrix _{S}^{E}R from the segment coordinate system <S> to the camera coordinate system <E> based on the third marker Mk3 included in the captured image IM2. Alternatively, the calibration part 180 may derive the positions and postures of the segments of the estimation target TGT by analyzing the captured image, thereby deriving the conversion matrix _{S}^{E}R from the segment coordinate system <S> to the camera coordinate system <E>. For example, the position and posture of the head among the segments may be estimated by a technique of estimating the face orientation from the feature points of the face. In this case, it is preferable that the image capturing device 50 may measure the distance like a time-of-flight (TOF) camera since it may obtain the three-dimensional contour of the estimation target TGT.

<Modified Example of Method of Obtaining Captured Image>

Hereinafter, a method of obtaining a captured image other than the method by using a drone will be described. FIG. 18 is a diagram for illustrating a (first) modified example of the method of obtaining the captured image. As shown in the figure, for example, one or more image capturing devices 50A may be attached to a gate or the like through which the estimation target TGT passes to obtain one or more captured images as the estimation target TGT passes. In this case, since the image capturing device 50A is stationary, the global coordinate system <G> and the camera coordinate system <E> may be equated. Therefore, the second marker Mk2 may be omitted even in the case where the third marker Mk3 is not present.

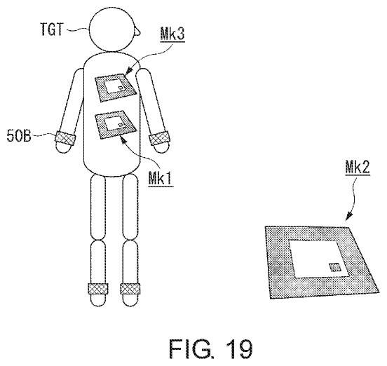

FIG. 19 is a diagram for illustrating a (second) modified example of the method of obtaining the captured image. As shown in the figure, for example, one or more image capturing devices 50B (micro camera rings) attached to a wristband or an ankle band may be attached to the estimation target TGT to obtain one or more captured images. In this case, it is preferable that the second marker Mk2 is present, and it is preferable that the estimation target TGT is instructed to take a predetermined pose when an image is captured by the image capturing device 50B.

Alternatively, one or more image capturing devices may be attached to the floor, the wall surface, the ceiling, or the like to obtain the captured images.

Although embodiments for implementing the disclosure have been described above by the embodiments, the disclosure is not limited to these embodiments, and various modifications and replacements may be added without departing from the spirit of the disclosure.

What is claimed is:

1. An analysis device comprising:
a posture estimation part which estimates a posture of an estimation target including a process of converting an output of a plurality of inertial measurement sensors expressed in a sensor coordinate system based on respective positions of the inertial measurement sensors that are attached to a plurality of sites of the estimation target and detect angular velocity and acceleration into a segment coordinate system expressing postures of respective segments corresponding to the positions where the inertial measurement sensors are attached in the estimation target;
an obtaining part which obtains an image captured by an image capturing part that captures an image of one or more first markers provided on the estimation target; and
a calibration part which calibrates a conversion rule from the sensor coordinate system to the segment coordinate system based on the image,
wherein the first marker has a form in which a posture relative to at least one of the inertial measurement sensors does not change, and the posture with respect to the image capturing part is recognizable by analyzing the captured image, and
the calibration part derives the posture of the first marker with respect to the image capturing part, derives a conversion matrix from the sensor coordinate system to a camera coordinate system based on the derived posture, and calibrates the conversion rule from the sensor coordinate system to the segment coordinate system by using the derived conversion matrix from the sensor coordinate system to the camera coordinate system.

2. The analysis device according to claim 1, wherein the image capturing part further captures an image of a second marker which is stationary in a space where the estimation target is present,
the second marker has a form in which a posture with respect to the image capturing part is recognizable by analyzing the captured image, and
the calibration part derives the posture of the second marker with respect to the image capturing part, derives a conversion matrix from a global coordinate system expressing the space to the camera coordinate system based on the derived posture, and equates the segment coordinate system with the global coordinate system, whereby the calibration part derives a conversion matrix from the sensor coordinate system to the segment coordinate system based on the conversion matrix from the sensor coordinate system to the camera coordinate system and the conversion matrix from the global coordinate system to the camera coordinate system and calibrates the conversion rule from the sensor coordinate system to the segment coordinate system based on the derived conversion matrix from the sensor coordinate system to the segment coordinate system.

3. The analysis device according to claim 1, wherein the image capturing part further captures an image of a third marker which is provided on the estimation target,
the third marker has a form in which a posture relative to at least one of the segments does not change, and the posture with respect to the image capturing part is recognizable by analyzing the captured image, and
the calibration part derives the posture of the third marker with respect to the image capturing part, derives a conversion matrix from the segment coordinate system to the camera coordinate system based on the derived posture, derives a conversion matrix from the sensor coordinate system to the segment coordinate system based on the conversion matrix from the sensor coordinate system to the camera coordinate system and the conversion matrix from the segment coordinate system to the camera coordinate system and calibrates the conversion rule from the sensor coordinate system to the segment coordinate system based on the derived conversion matrix from the sensor coordinate system to the segment coordinate system.

4. The analysis device according to claim 2, wherein the image capturing part further captures an image of a third marker which is provided on the estimation target,
the third marker has a form in which a posture relative to at least one of the segments does not change, and the posture with respect to the image capturing part is recognizable by analyzing the captured image, and
the calibration part derives the posture of the third marker with respect to the image capturing part, derives a conversion matrix from the segment coordinate system to the camera coordinate system based on the derived posture, derives a conversion matrix from the sensor coordinate system to the segment coordinate system based on the conversion matrix from the sensor coordinate system to the camera coordinate system and the conversion matrix from the segment coordinate system to the camera coordinate system and calibrates the conversion rule from the sensor coordinate system to the segment coordinate system based on the derived conversion matrix from the sensor coordinate system to the segment coordinate system.

5. An analysis method, wherein a computer performs:
estimating a posture of an estimation target including a process of converting an output of a plurality of inertial measurement sensors expressed in a sensor coordinate system based on respective positions of the inertial measurement sensors that are attached to a plurality of sites of the estimation target and detect angular velocity and acceleration into a segment coordinate system expressing postures of respective segments corresponding to the positions where the inertial measurement sensors are attached in the estimation target;
obtaining an image captured by an image capturing part that captures an image of one or more first markers provided on the estimation target; and
calibrating a conversion rule from the sensor coordinate system to the segment coordinate system based on the image,
wherein the first marker has a form in which a posture relative to at least one of the inertial measurement sensors does not change, and the posture with respect to the image capturing part is recognizable by analyzing the captured image, and
in the process of calibrating, the computer derives the posture of the first marker with respect to the image capturing part, derives a conversion matrix from the sensor coordinate system to a camera coordinate system based on the derived posture, and calibrates the conversion rule from the sensor coordinate system to the segment coordinate system by using the derived conversion matrix from the sensor coordinate system to the camera coordinate system.

6. A non-transient computer-readable recording medium, recording a program which makes a computer perform:
estimating a posture of an estimation target including a process of converting an output of a plurality of inertial measurement sensors expressed in a sensor coordinate system based on respective positions of the inertial measurement sensors that are attached to a plurality of sites of the estimation target and detect angular velocity and acceleration into a segment coordinate system expressing postures of respective segments corresponding to the positions where the inertial measurement sensors are attached in the estimation target;
obtaining an image captured by an image capturing part that captures an image of one or more first markers provided on the estimation target; and
calibrating a conversion rule from the sensor coordinate system to the segment coordinate system based on the image,
wherein the first marker has a form in which a posture relative to at least one of the inertial measurement sensors does not change, and the posture with respect to the image capturing part is recognizable by analyzing the captured image, and
in the process of calibrating, the computer derives the posture of the first marker with respect to the image capturing part, derives a conversion matrix from the sensor coordinate system to a camera coordinate system based on the derived posture, and calibrates the conversion rule from the sensor coordinate system to the segment coordinate system by using the derived conversion matrix from the sensor coordinate system to the camera coordinate system.

7. A calibration method comprising:
capturing an image of the one or more first markers provided on the estimation target by the image capturing part equipped on an unmanned aerial vehicle; and
obtaining the image captured by the image capturing part and calibrating the conversion rule from the sensor coordinate system to the segment coordinate system by the analysis device according to claim 1.

8. A calibration method comprising:
capturing an image of the one or more first markers provided on the estimation target by the image capturing part equipped on an unmanned aerial vehicle; and
obtaining the image captured by the image capturing part and calibrating the conversion rule from the sensor coordinate system to the segment coordinate system by the analysis device according to claim 2.

9. A calibration method comprising:
capturing an image of the one or more first markers provided on the estimation target by the image capturing part equipped on an unmanned aerial vehicle; and
obtaining the image captured by the image capturing part and calibrating the conversion rule from the sensor coordinate system to the segment coordinate system by the analysis device according to claim 3.

10. A calibration method comprising:
capturing an image of the one or more first markers provided on the estimation target by the image capturing part equipped on an unmanned aerial vehicle; and
obtaining the image captured by the image capturing part and calibrating the conversion rule from the sensor coordinate system to the segment coordinate system by the analysis device according to claim 4.

11. A calibration method comprising:
capturing an image of the one or more first markers provided on the estimation target by the image capturing part attached to a stationary object; and
obtaining the image captured by the image capturing part and calibrating the conversion rule from the sensor coordinate system to the segment coordinate system by the analysis device according to claim 1.

12. A calibration method comprising:
capturing an image of the one or more first markers provided on the estimation target by the image capturing part attached to a stationary object; and
obtaining the image captured by the image capturing part and calibrating the conversion rule from the sensor coordinate system to the segment coordinate system by the analysis device according to claim 2.

13. A calibration method comprising:
capturing an image of the one or more first markers provided on the estimation target by the image capturing part attached to a stationary object; and
obtaining the image captured by the image capturing part and calibrating the conversion rule from the sensor coordinate system to the segment coordinate system by the analysis device according to claim 3.

14. A calibration method comprising:
capturing an image of the one or more first markers provided on the estimation target by the image capturing part attached to a stationary object; and
obtaining the image captured by the image capturing part and calibrating the conversion rule from the sensor coordinate system to the segment coordinate system by the analysis device according to claim 4.

15. A calibration method comprising:
capturing an image of the one or more first markers provided on the estimation target by the image capturing part attached to the estimation target; and
obtaining the image captured by the image capturing part and calibrating the conversion rule from the sensor coordinate system to the segment coordinate system by the analysis device according to claim 1.

16. A calibration method comprising:
capturing an image of the one or more first markers provided on the estimation target by the image capturing part attached to the estimation target; and
obtaining the image captured by the image capturing part and calibrating the conversion rule from the sensor coordinate system to the segment coordinate system by the analysis device according to claim 2.

17. A calibration method comprising:
capturing an image of the one or more first markers provided on the estimation target by the image capturing part attached to the estimation target; and
obtaining the image captured by the image capturing part and calibrating the conversion rule from the sensor coordinate system to the segment coordinate system by the analysis device according to claim 3.

18. A calibration method comprising:
capturing an image of the one or more first markers provided on the estimation target by the image capturing part attached to the estimation target; and
obtaining the image captured by the image capturing part and calibrating the conversion rule from the sensor coordinate system to the segment coordinate system by the analysis device according to claim 4.
